(19) United States
(12) Patent Application Publication
(10) Pub. No.: US 2022/0375557 A1
(43) Pub. Date: Nov. 24, 2022

(54) DATA PROCESSING AND HUMAN INTERFACE SYSTEM FOR ADMISSION AND DISCHARGE MANAGEMENT

(71) Applicant: Clover Health Investments, Corp., Franklin, TN (US)

(72) Inventors: Calvin Chock, Danville, CA (US); Molly Inglish, Atlanta, GA (US); William Masson, Oakland, CA (US); Mark Spektor, Lawrence Township, NJ (US); Earl Bryant, Bradenton, FL (US)

(21) Appl. No.: 17/750,111

(22) Filed: May 20, 2022

Related U.S. Application Data
(60) Provisional application No. 63/191,182, filed on May 20, 2021.

(51) Int. Cl. G16H 10/60; G16H 40/60

(52) U.S. Cl. CPC G16H 10/60; G16H 40/60; G16H 40/63

(57) ABSTRACT

A method, apparatus, and system for assisting a user with managing patient conditions, treatments, admissions, and discharges may include at least one processor coupled to an electronic memory, a network interface, and a display. The memory holds instructions that when executed by the processor cause an apparatus to perform displaying on a display of a client device an identifier of a patient in a list of patients from a patient database, outputting a first input object on the client device for user input indicating health conditions of the patient, outputting a second input object on the client device for user input confirming at least one of an admission status of the patient or a discharge status of the patient, and providing to the patient database an identifier of the selected patient, the user input indicating the health conditions and the admission status or the discharge status of the patient.

### PRIORITY CLAIM

The present application claims priority to U.S. provisional patent application Ser. No. 63/191,182 filed May 20, 2021, which is incorporated herein in its entirety by reference.

### FIELD

The present application relates to systems, apparatus or methods for routing and presenting data, receiving inputs, and managing responses to inputs, for application in managing admission and discharge of patients to hospitals or similar health care institutions.

### BACKGROUND

Doctors and other health care providers often organize into practices to care for patients. The practice may include various specialties and skills, while the primary medical contact for the patient is the Primary Care Provider (PCP). Primary care providers are concerned with managing patients' ongoing care. To that end, if a patient is admitted into the emergency room, urgent care facility, or other facility independently of the PCP, the PCP benefits from receiving information about the independent visits because it allows them to tailor their care to the patient's needs. For example, many PCPs will do rounds at hospitals, including deciding admission types and appropriate post-acute care, to effectively manage the health needs of patients under their care.

In some cases practices employ Hospitalists that are dedicated to seeing patients in hospitals. These Hospitalists act on behalf of the PCP and similarly guide the patient's care. Hospitalists can have privileges at one or many hospitals and often make care decisions for multiple patients in any given hospital. These Hospitalists act as go-between between the hospital practitioners and one or many PCPs.

Hospitalists may organize into groups. Hospitalist groups are often employed by practices to provide care to beneficiaries in a hospital. Hospitalists in these groups act as a go-between between a practice and a hospital. In general, Hospitalists are compensated by the practice and will comply with a practice's preferred operating procedure. Hospitalists will similarly support decision making around a patient's care needs when in a hospital.

A critical point of care for patients occurs at admission or discharge from a hospital. Hospitalists that support beneficiaries in the Emergency Room as these patients progress towards admission to the hospital or as they near discharge from the hospital. When making an admission or discharge decision for a beneficiary being either admitted or prepared for discharge, Hospitalists consider a large set of criteria when deciding if an admission should be for Observation or In-Patient or, in the case of a discharge, what preferred locations to recommend. Often they must make such decisions without consulting with the PCP, who is not easily informed about the reasons for specific decisions.

When making an admission or discharge decision for a beneficiary being either admitted or prepared for discharge, doctors have to consider numerous criteria when deciding if an admission should be for Observation or In-Patient or, in the case of a discharge, what preferred locations to recommend. While hospitals and practice groups use database tools for patient records, such tools are not optimized for use in managing hospital admission and discharge. Thus, management of admission and discharge is subject to unnecessary inconsistencies and inefficiencies.

It would be desirable, therefore, to develop new tools, methods and other new technologies for managing admission and discharge of patients to hospitals or similar health care institutions, that overcomes these and other limitations of the prior art.

### SUMMARY

This summary and the following detailed description should be interpreted as complementary parts of an integrated disclosure, which parts may include redundant subject matter and/or supplemental subject matter. An omission in either section does not indicate priority or relative importance of any element described in the integrated application. Differences between the sections may include supplemental disclosures of alternative embodiments, additional details, or alternative descriptions of identical embodiments using different terminology, as should be apparent from the respective disclosures.

In an aspect of the disclosure, a method for configuring a client device to assist a user with managing patient conditions, treatments, admissions, and discharges may include displaying, by at least one processor on a display of a client device, an identifier of a patient in a list of patients from a patient database. The method may include outputting a first input object on the client device for user input indicating health conditions of the patient and outputting a second input object on the client device for user input confirming at least one of an admission status of the patient or a discharge status of the patient. The method may further include providing an identifier of the selected patient, the user input indicating the health conditions, and the at least one of an admission status or the discharge status of the patient.

In related aspects, the method may include determining that an admission status of the patient on a watchlist for the client device has changed and outputting an admissions alert on the client device identifying the patient and the status change. Optionally, the determining and the outputting the admissions alert are preconditions to performing all steps of the method summarized in the foregoing paragraph.

The method may further include displaying details of patient conditions at time of admission to a healthcare facility or at time of discharge from the healthcare facility.

The method may further include displaying on the client device a list of completed admission alerts by the user of the client device. In an aspect, the client device may omit or obscure ones of the completed admission alerts that are aged more than a defined maximum time interval (e.g., 24, 48 or 72 hours) after creation from display on the client device.

In an aspect, the first input object may be, or may include, a checklist for admission of the patient to a healthcare facility, and further comprising, by the at least one processor, causing the client device to display an indication that an admissions alert is in a pending status. In a related aspect, the method may include receiving an indication that the patient is discharged from the healthcare facility and closing the admissions alert in response. The method may include sending a message to a specified electronic address indicating that a new admissions alert is in a pending state. The method may include configuring the checklist based on a diagnosis record for the patient. In an aspect, the configuring may include providing alternative admissions criteria in the checklist for display on the client device. The method may include determining a recommendation regarding whether the patient should be admitted to a healthcare facility in response to completion of the checklist by the user of the client device.

The method may include filtering the list of patients by a user input indicating one or more healthcare facilities. In an aspect, the user input may be performed by a different user using a different device in communication with the client device.

The method may include configuring at least one of an alert frequency and type in response to user input on the client device.

The method may include receiving a user input from the client device indicating a selected patient from the list of patients and placing an identifier for the selected patient on a watchlist for the client device.

The method may include presenting a checklist of discharge criteria concerning the patient for completion by the user of the client device, and based on completion of the checklist, selecting an indication of an appropriate discharge location type for display on the client device. In an aspect, the appropriate discharge location type may be based at least in part on a geographic location of the patient.

An apparatus for assisting a user with managing patient conditions, treatments, admissions, and discharges may include a processor coupled to an electronic memory, the memory holding instructions that when executed by the processor cause the apparatus to perform operations of the method summarized above. In a related aspect, a system for assisting a user with managing patient conditions, treatments, admissions, and discharges may include the apparatus, a client database, a system management server, and other components.

As used herein, a “client device” includes at least a computer processor coupled to a memory and to one or more ports, including at least one input port and at least one output port or display device (e.g., a desktop computer, laptop computer, tablet computer, smartphone, PDA, etc.). A “mobile device” is a mobile client device, for example, a smartphone. A computer processor may include, for example, a microprocessor, microcontroller, system on a chip, or other processing circuit. As used herein, a “processor” means a computer processor.

To the accomplishment of the foregoing and related ends, one or more examples comprise the features hereinafter fully described and particularly pointed out in the claims. The following description and the annexed drawings set forth in detail certain illustrative aspects and are indicative of but a few of the various ways in which the principles of the examples may be employed. Other advantages and novel features will become apparent from the following detailed description when considered in conjunction with the drawings and the disclosed examples, which encompass all such aspects and their equivalents.

### BRIEF DESCRIPTION OF THE DRAWINGS

The features, nature, and advantages of the present disclosure will become more apparent from the detailed description set forth below when taken in conjunction with the drawings in which like reference characters identify like elements correspondingly throughout the specification and drawings.

FIG. 1 is a schematic diagram illustrating a system for assisting one or more users in managing admission and discharge of patients to hospitals.

FIG. 2 is a functional block diagram illustrating components of the system for managing admission and discharge of patients to hospitals.

FIG. 3 is a flow diagram illustrating a view of process flow in the system for managing admission and discharge of patients to hospitals.

FIG. 4 is a flow diagram illustrating more detailed functions and aspects related to the process flow of FIG. 3.

FIG. 5A is a screenshot illustrating an example of patients with pending admissions status, also called a “to-do” list, for display on a client device of the system.

FIG. 5B is a screenshot illustrating an example of a notification screen signaling completion of a to-do list, for display on a client device of the system.

FIG. 6 is a screenshot illustrating an example of a patient conditions checklist, for display on a client device of the system.

FIG. 7 is a screenshot illustrating further aspects of the example of a patient conditions list, for display on a client device of the system.

FIG. 8 is a screenshot illustrating an example of an admissions recommendation screen recommending observation status, for display on a client device of the system.

FIG. 9 is a screenshot illustrating an example of an admissions recommendation screen recommending inpatient status, for display on a client device of the system.

FIG. 10 is a screenshot illustrating an example of a discharge details screen, for display on a client device of the system.

FIG. 11 is a screenshot illustrating an example of a list of facilities for discharge, for display on a client device of the system.

FIG. 12 is a screenshot illustrating an example of a metadata screen for a selected discharge facility, for display on a client device of the system.

FIG. 13A is a screenshot illustrating an example of a patient metadata screen, for display on a client device of the system.

FIG. 13B is a screenshot illustrating an example of a provider/doctor/user metadata display screen, for display on a client device of the system.

FIG. 14 is a flow chart illustrating aspects of a method for configuring a client device to assist a user with managing patient conditions, treatments, admissions, and discharges.

FIGS. 15-17 are flow charts illustrating further aspects of the method of FIG. 14.

FIG. 18 is a conceptual block diagram illustrating components of an apparatus or system for configuring a client device to assist a user with managing patient conditions, treatments, admissions, and discharges.

### DETAILED DESCRIPTION

Various aspects are now described with reference to the drawings. In the following description, for purposes of explanation, numerous specific details are set forth to provide a thorough understanding of one or more aspects. It may be evident, however, that the various aspects may be practiced without these specific details. In other instances, well-known structures and devices are represented in block diagram form to facilitate focus on novel aspects of the present disclosure.

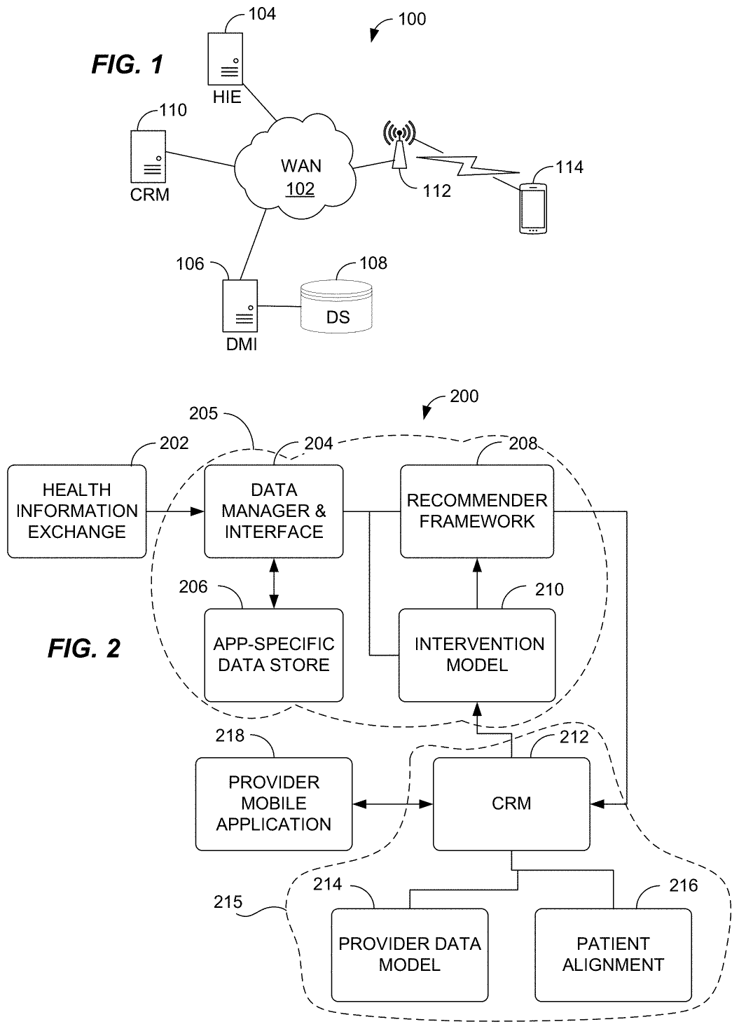

FIG. 1 shows an example of a system 100 for assisting one or more users in managing admission and discharge of patients to hospitals. The system 100 may include several servers 104, 106, 110 connected to each other and to a client device 114 (e.g., a smartphone, laptop, desktop, or notebook computer) via a Wide Area Network 102 (e.g., the Internet). Optionally, the client device 114 may be connected to the servers 104, 106, 110 via the WAN 102 and a wireless link 112, for example, a local area network (LAN) that includes a wireless router/modem, or a cellular or satellite telephone network. The servers 104, 106, 110 may be instantiated by a single server machine, by an array or farm of interconnected servers on a network, by rented resources on a cloud server, or in any other suitable fashion. The servers 104, 106, 110 may be independently managed and communicate with each other through one or more application program interfaces (APIs) and security protocols.

The system 100 may be used to service a solution to managing Admission, Discharge and Transfer (ADT) including a mobile ‘Inpatient Administration’ (IA) application 218 (FIG. 2) for use by a Hospitalist or PCP of a medical practice group on a mobile device. Configuration and operation of the mobile IA application 218 may be managed via a data management interface component 205, optionally with the help of an account administrator with access to the medical practice group's patient and provider data maintained by the CRM server 110 running a CRM application 212. The account administrator may implement policy of the practice group's insurance administrator to improve consistency and effectiveness of a member hospital's ADT protocol.

The mobile IA application 218 may alert a Hospitalist or PCP when one of their patients has been admitted into the emergency room. The IA application 218 may then guide the Hospitalist or PCP through a patient list and present the option to complete an “admissions checklist” which will provide a recommendation for in-patient admission or observation based on the selected condition and present symptoms identified by the user in the checklist. Additionally, the application may direct the Hospitalist to review discharge options based on the patient's needs. The “discharge checklist” presented on the client device 114 may guide the Hospitalist through a series of questions and recommend a discharge type based on the criteria in the checklist. Further, the IA application 218 may recommend a preferred discharge location that is conveniently located near the beneficiary.

The mobile IA application 218 may support native client device 114 alerts and in-app alerts, for example, email and SMS alerts that the user (e.g., a Hospitalist or PCP) can receive and send on the client device 114. Using the mobile IA application 218 operating on the client device 114, the user can manage their contact information and alert preferences. The mobile IA application 218 may be distributed as a commercial mobile application, for example, via iOS and Android app stores, or privately via a custom URL.

Referring to both FIGS. 1 and 2, the Health Information Exchange (HIE) Server 104 holds information on the patient, including medical records exchanged between medical providers. HIE refers to electronic exchange of patients' healthcare-related data among medical facilities, health information organizations and government agencies according to standards by state or federal governing bodies. For example, in the United States, the ONC (Office of the National Coordinator for Health Information Technology) created the Nationwide Health Information Network (NHIN) to establish standards, services and policies for HIE. Federal agencies, HIEs and healthcare providers agreed to adopt NHIN standards for secure HIE at a local and national level. NHIN was renamed the eHealth Exchange in 2012.

Examples of functions performed by components of the system 100 are illustrated by FIG. 2 in a corresponding software system 200. The function of the HIE server 104 may include maintaining and securing medical data 202 in compliance with regulatory standards, and communicating with authorized medical providers, insurers, and other service providers to facilitate use and updating of patient records.

The HIE server 104 communicates with the Data Manager and Interface (DMI) server 106 that implements communication with the client device and coordination with data from the Customer Relationship Management (CRM) server 110. The DMI server 106 executing a DMI server application 205 may store data generated from interactions with a user via a client device 114, with the HIE server 104, and with the CRM server 110 executing CRM application 215 in a local data store 108, or in a remote storage device, cloud, or peer-to-peer network. Functionally, the DMI server application 205 may include an application-specific data storage component 206 servicing a data manager and interface component 204, which drives the user experience via a provider mobile IA application 218 operating on the client device 114. The CRM application 215 may exchange data directly with the mobile IA application 218 and with the DMI application 205 via its intervention model module 210 and recommender framework 208. The intervention model module 210 applies the system 100 operator's policies and tools for managing ADT, for example by configuring ADT checklists. The recommender framework module 208 generates a recommended action, and in the case of discharge, one or more discharge facility options.

The CRM application 215 receives recommendations from the recommender module and passes them to the user via the mobile IA application 218. The CRM application 215 may use a patient alignment module 216 to determine the identity and electronic address of the user responsible for the patient's care when an ADT determination or interaction is needed. Discharge locations may be categorized by type in a CRM model for the storing and management of location data for use in interacting with the mobile IA application 218 or DMI application 205. The CRM application 215 may include a provider data model module 214 model for the storing and management of user (e.g., Hospitalist/PCP) data to fulfill the needs of the DMI application 205. The model may, for example, store and manage health care providers (e.g., hospitalists and PCPs) by practice group with the hospitals where each provider has privileges.

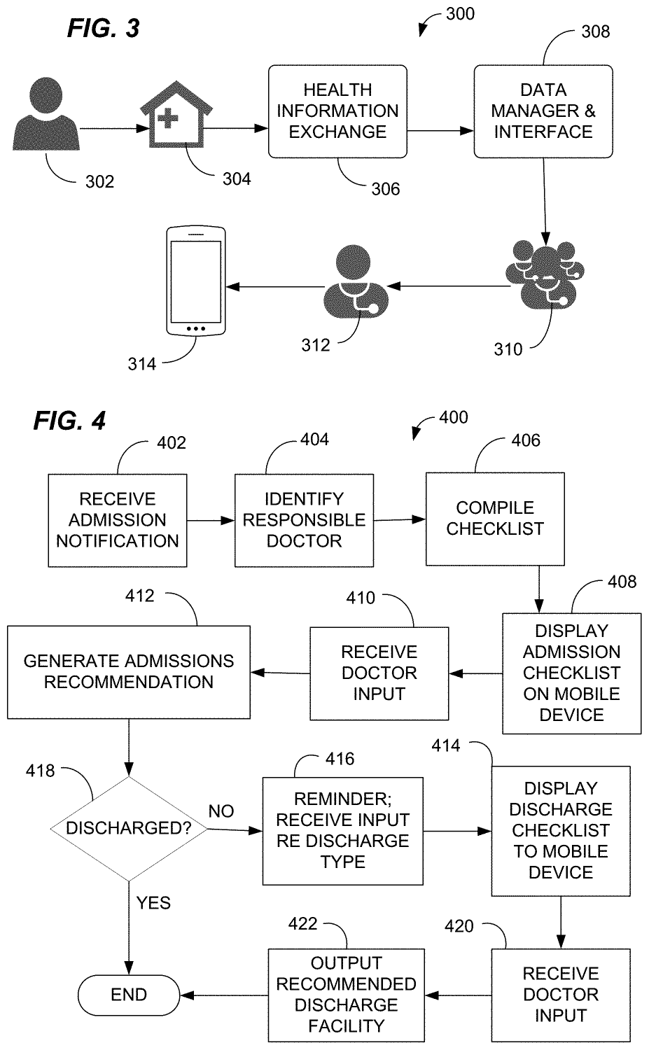

FIG. 3 shows an example of process flow 300 in the system 100 for managing admission and discharge of patients to hospitals. Admission of a patient 302 to a hospital 304 triggers initiation of the process 300, when as part of ordinary admissions processing, the hospital 304 sends an ADT alert to the health information exchange (HIE) system 306 immediately after inpatient processing by hospital staff. The HIE system 306 alerts the data manager and interface (DMI) server 308 that the patient 302 is admitted to the hospital 304. In the flow diagram of FIG. 4, a DMI process 400 for the patient 302 initiates at block 402, when the DMI component 308 receives the admission alert from the HIE system.

Referring FIGS. 3 and 4, the DMI server 308 identifies the doctor 404 responsible for managing admission of the patient 302 to the hospital 304, by executing an algorithm. The algorithm may include, for example, identifying the practice group 310 to which the patient 302 belongs and the hospitalist 312 serving the hospital 304, by communicating with the internal data store 108 and/or the CRM server 110 for the practice group 310.

The DMI server sends an alert to a mobile IA application 218 operating on the mobile device 314, indicating an admissions status of patient 302 in the hospital 312. The mobile device 314 outputs an alert for the hospitalist to which it belongs. For example, the mobile device may chime or vibrate, and output an alert message on its screen. The alert message may include a link to initiate a login session, enabling the hospitalist user to authenticate identity and view the details of the admission of the patient 302. Once the user is authenticated, the DMI server may send data to the mobile application for populating a “to-do” list of pending ADT events needing the hospitalist's input.

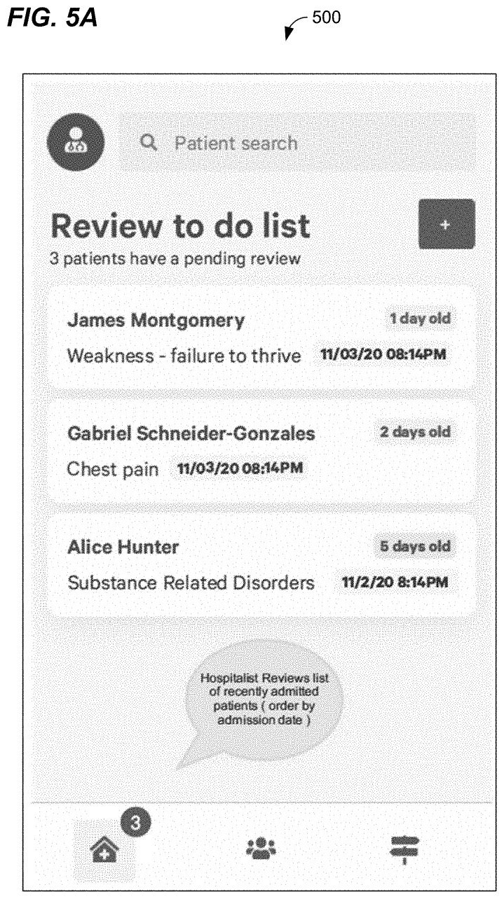

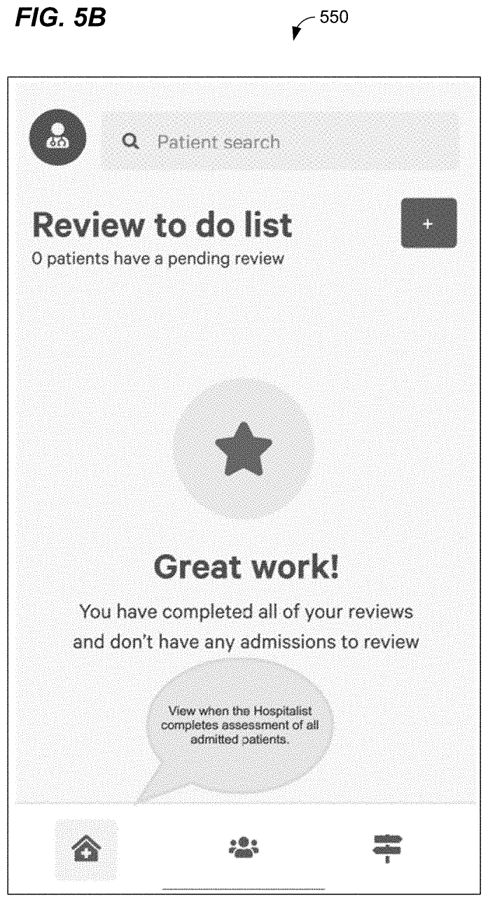

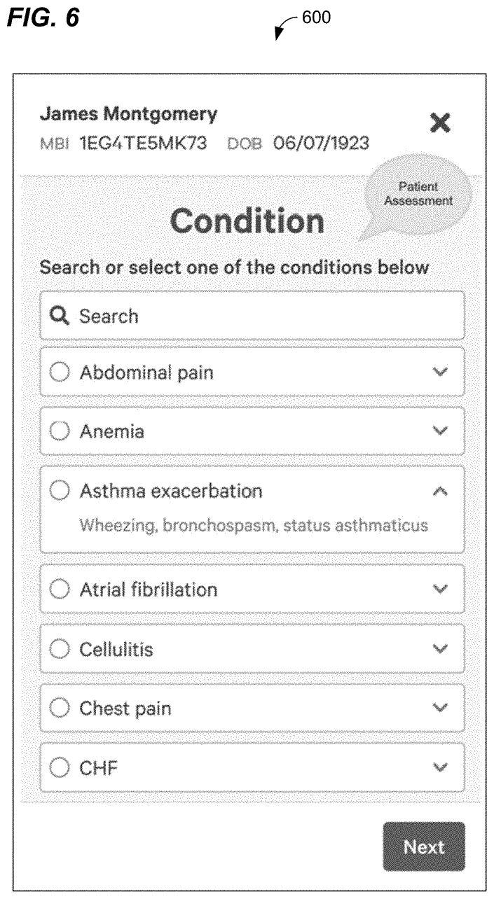

The mobile IA application 218 operating on the mobile device 314 may use the pending ADT list to populate a “to-do” list. For example, the mobile IA application 218 may output a “to do” list style screen 500, as shown in FIG. 5A. The screen 500 illustrates a to-do list more than 24 hours after patient admission. For a more recent admission, the to-do list may be configured to place identifiers for the patient 203 (not shown in FIG. 5A) at the top of the list. In the example screen 500, the display includes next to each patient identifier in the list, the time period since last ADT status change, a date and time of the change in ADT status, and an indicator of the patient's condition. Once the doctor has provided input completing assessment for all patients in the “to-do” screen 500, the mobile device 314 may present an empty list 550, for example as shown in FIG. 5B. The comments provided in a speech bubble in screens 5A-B comprise information for the present reader only and are not intended to indicate text for display on the screen during operation of the process 400. Similar comments placed in the example screenshots of FIGS. 6 and 8-10 are likewise for information of the present reader only.

In response to user input to the mobile application indicating a user selection of a patient in the “to-do” list 500, the DMI server 308 and/or mobile device 314 may compile an admissions checklist 406. In an aspect, the server 308 may populate a data table object with values extracted from one or more of: data from the HIE server 104, data from the CRM server 110, and data from its own data store 108. The server may send the data table object to the mobile device 114, which is executing the mobile IA application 218. The mobile IA application 218 may receive the data object and insert it into a container object, e.g., an XTML page configured for display in a browser of the mobile device, or directly by the IA application 218.

The DMI server 308 may generate the checklist in phases, beginning with a list of general health conditions that are observed in the admitted patient, for first-level screening of the patient. Optionally, the DMI server may customize the checklist based on the patient's gender, age, prior health status, or other information in its data store. In an alternative, or in addition, the mobile device 314 may retrieve a predetermined dataset for generating a first-level checklist from its local memory. The mobile application may display the admission checklist values 408 in a graphical user interface screen or other interface that is enabled for entry of user data. In response to output of the admissions checklist, the hospitalist user may enter data into a form or other interface of the mobile IA application 218.

FIG. 6 shows an example of a first-level admissions checklist 600 for output on the display of the mobile device 314. The checklist 600 may be configured so that the hospitalist can scroll through the condition list, or search for a specific condition. Using the togglable selection object displayed next to each item in the condition list, the user selects the applicable conditions observed by the user to be present in the patient. The user may finalize the selections for the mobile application by activating an interactive object on the screen (not shown in checklist 600) indicating that the user is finished with selecting. The mobile device 314 and IA application 218 may receive the input 410 and generate a message to the DMI server 308 reporting the doctor input.

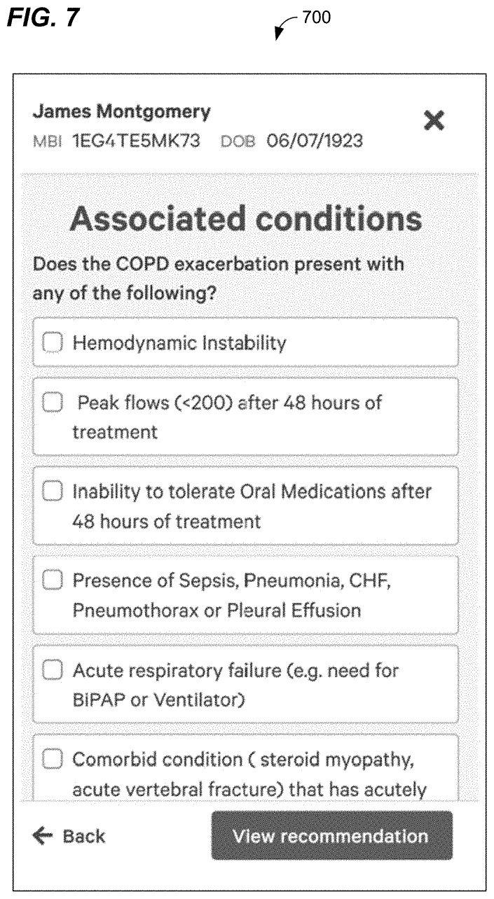

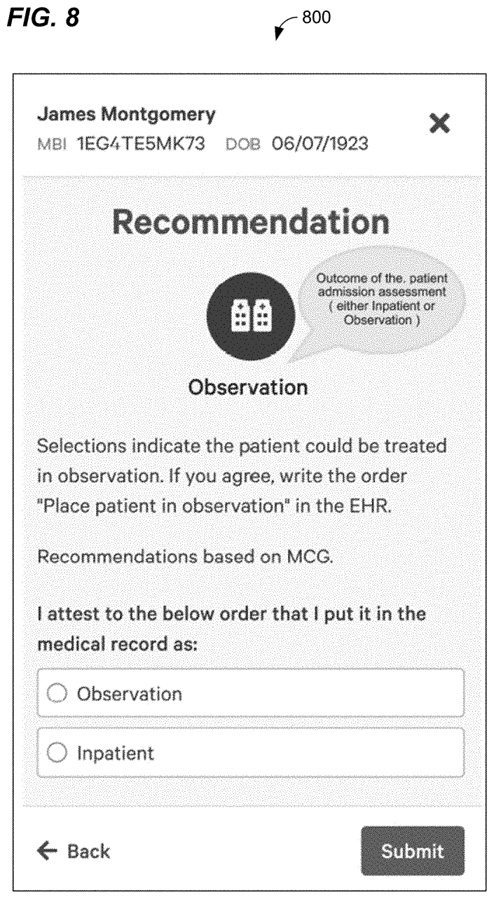

In an aspect, the DMI server 308 and/or mobile device 314 may generate a next-level condition list, in response to selection of any condition on a prior list. The next-level condition list may be configured to request more detailed information regarding an observed condition. For example, FIG. 7 shows an associate conditions list 700, requesting more detailed information form the user regarding a specific condition (in this example, the specific condition is COPD). The user may select from the list and activate an interactive object on the screen, e.g., the button labeled “View recommendation,” indicating that the user is finished with selecting. The mobile IA application 218 may receive and retransmit the user input as previously described for screen 600.

Referring again to FIG. 4 at 412, the DMI server and/or mobile device may generate an admissions recommendation based on completed user input to the admission checklists. Various expert systems, including decision trees or artificial intelligence, may be used by the DMI server to generate a recommendation based on the user input. For a useful recommendation, ADT data should be received within a reasonable time window to allow the hospitalist user ample time to address the patient's admissions needs. ADT data should be consistently complete and reliable to allow for the automation of workflows to limit the need for manual data management and review. In addition, ADT quality and timeliness should be consistent across markets so that one set of automations can be delivered systemwide.

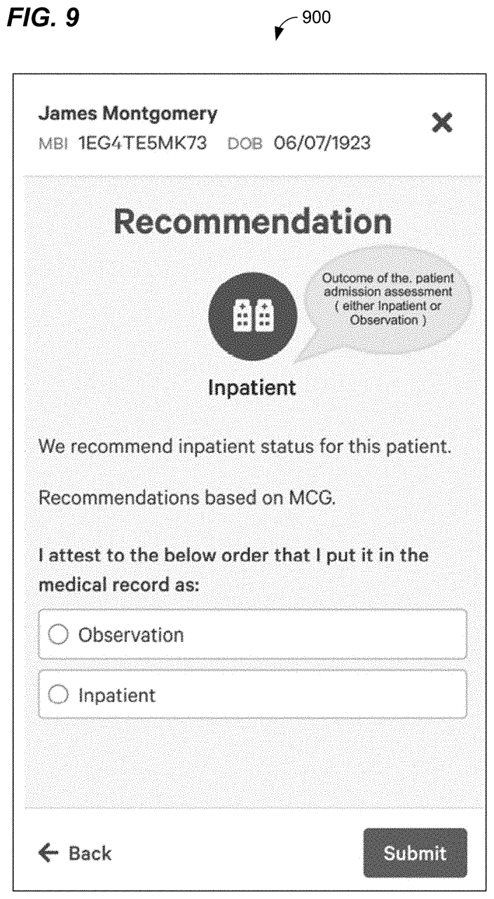

Once the recommendation is determined and provided to the mobile IA application 218, the mobile device may display the recommendation on its display screen. FIG. 8 shows an example of an admissions recommendation screen 800. In this example, the screen 800 displays a recommendation for observation status. Being a less intensive status, costs of observation status are less than inpatient status, and it is beneficial to the system to avoid unnecessary inpatient admissions. FIG. 9 shows a recommendation screen 900 that contains a recommendation for inpatient status. The screens 800 or 900 may be configured to enable the user to confirm the recommendation or to indicate a contrary opinion, for example, inpatient status, by selection one of two or more options. The selected decision can be received by the mobile device and provided to the DMI server as previously described. Once the status is determined by the user to be “observation” or “inpatient,” by the hospitalist user, activity for the admitted patient is suspended until the patient's AT status changes. Referring to FIG. 4, if the patient is discharged home, the HIE server will provide notice to the DMI server. If the DMI server received a notice that the patient is discharged at 418, the process 400 for managing admission and discharge of the patient may terminate. If the patient is not discharged after a set period (e.g., 24 hours, or 48 hours) the DMI application 205 and/or mobile IA application 218 may configure, generate and output on the mobile device 314 a discharge reminder 416 similar to screen 500 shown in FIG. 5A, listing patients that should be reviewed for discharge.

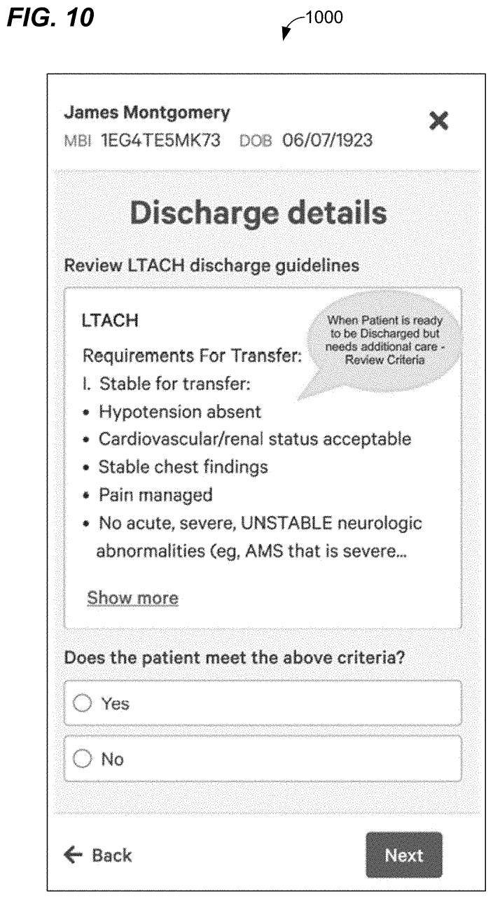

In response to selection of a particular patient on a discharge reminder list, and upon making a determination that the patient is ready to be discharged but needs additional care, the DMI application 205 and/or mobile IA application 218 may configure, generate and output on the mobile device 416 a discharge confirmation screen 1000, for example as shown in FIG. 10. The confirmation screen 100 lists recommended health status criteria for discharge of the patient from the hospital and provides input objects for the hospitalist user to indicate whether the patient indicated at the top of screen 1000 meets the recommended criteria. Via the input objects contained on this screen 1000, the mobile device 314 may receive user input indicating that the patient meets criteria for discharge. Upon receiving this input, the DMI application 205 and/or mobile IA application 218 may determine based on the patient's condition and/or user input whether the patient needs additional care.

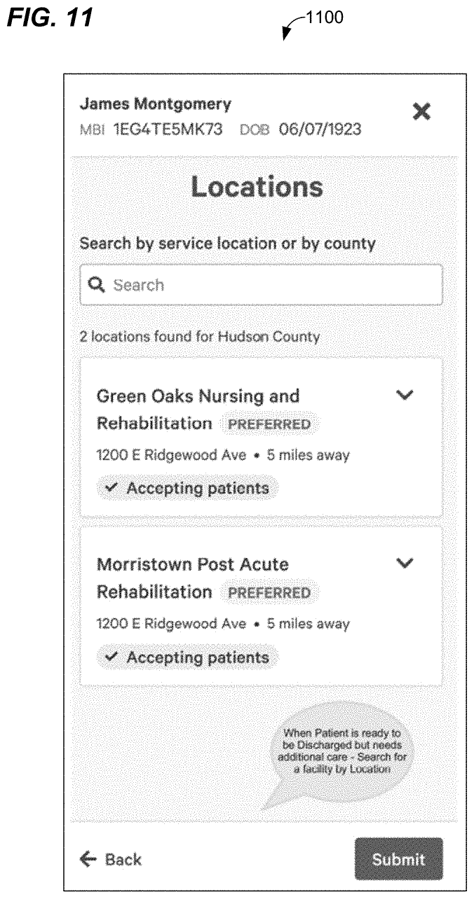

If it is determined that the patient needs additional care to be provided by a non-hospital facility (e.g., a nursing home or rehabilitation clinic), the DMI application 205 and/or mobile IA application 218 may generate a list of one or more discharge facilities 1100, for example as shown in FIG. 11, for display by the mobile device 314. The screen 1100 may contain a list of discharge facilities, and may indicate for each facility a name, address, preference status, and current availability. The screen 1100 may also include a search input enabling the user to search for a specific facility, whether listed or not. The screen may include scrolling features for paging or scrolling through a list longer than can fit in the display area.

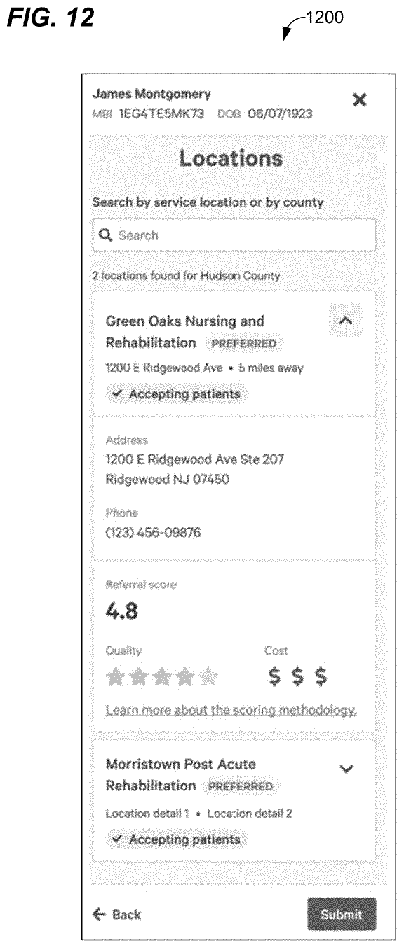

In response to user selection of a facility on the list, the DMI application 205 and/or mobile IA application 218 may output a facility information screen 1200, for example as shown in FIG. 12. The information screen 1200 may include the same information as shown for each facility on the list screen 1100, and in addition, a quality and cost rating for the facility and any other available information to assist the hospitalist user with selection of an appropriate facility. Referring again to FIG. 4 at 420, the mobile IA application 218 may receive user input from an interactive screen such as screens 1100 or 1200. At 422, the mobile IA application 218 may output information regarding the selected facility, for example as shown in FIG. 12, intended to be useful for coordinating the patient's discharge with hospital administrative staff. The process 400 may terminate once the discharge status is confirmed by the user. During the process 400, the DMI server 106 may collect data concerning user input and the context in which the input was received, for storing in its data store 108.

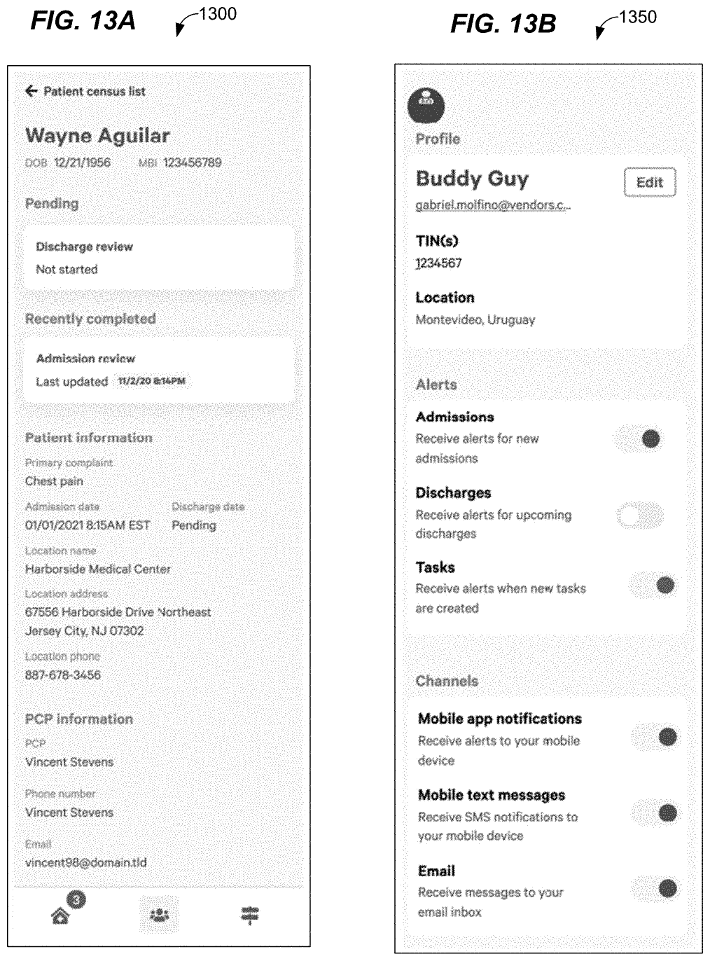

While not required for the process flow 400, the DMI application 205 and/or mobile IA application 218 may generate and output a patient metadata screen 1300 for display on a client device of the system, for example as shown in FIG. 13A. The patient metadata screen 1300 may include, for example, patient identifying information, ADT status, recent history of status updates, observed health conditions, a hospital identifier, and a PCP identifier with contact information.

Likewise, the DMI application 205 and/or mobile IA application 218 may generate and output a provider/doctor/user metadata display screen 1350 for display on a client device of the system, for example as shown in FIG. 13B. The provider/doctor/user metadata display screen 1350 may include, for example, an identifier of the user with current identifying and contact information, a location, the user's alert preferences, and the user's preferred communication channels. The user's preferences may be configured to be settable from the screen 1350, for example by using a toggling input object to indicate inclusion or omission of each preference item listed.

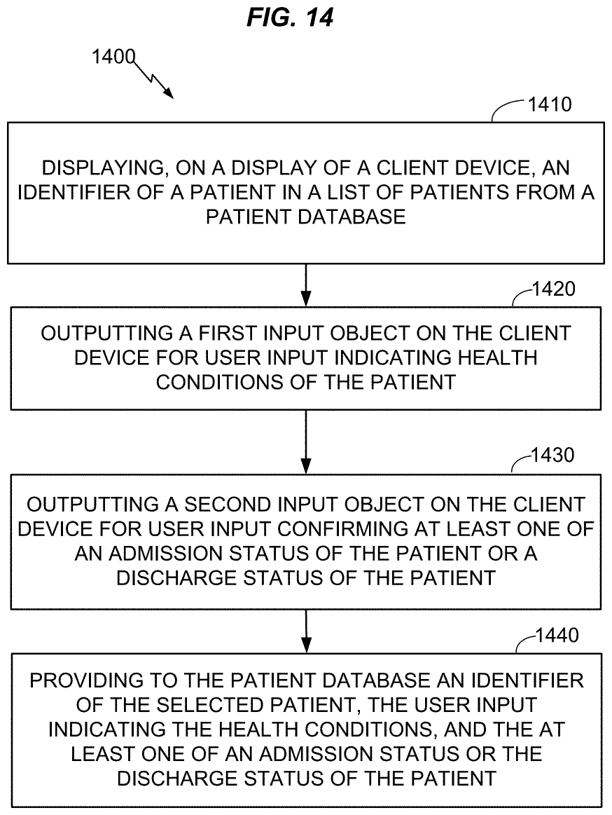

In accordance with the foregoing, and by way of additional example, FIG. 14 shows more general aspects of a method or methods 1400 for configuring a client device to assist a user with managing patient conditions, treatments, admissions, and discharges according to one embodiment, as may be performed by a DMI server as described herein, in communication with other processing nodes of the system 100 shown in FIG. 1. Operations of the method 1400 may include, embody or participate in more detailed aspects of corresponding operations described herein above and below and performed by the DMI server and other nodes of the system 100. Reference to “at least one processor” in connection with the description of FIGS. 14-17 below refers to at least one processor of the client device, alone or in combination with at least one processor of the DRM server and/or CRM server described herein above.

Referring to FIG. 14, a computer-implemented method for controlling a client device to assist a user with managing patient conditions, treatments, admissions, and discharges may include, at 1410 by at least one processor, displaying, on a display of a client device, an identifier of a patient in a list of patients from a patient database. For example, a DMI server may send an updated data object to the client device, wherein the updated data object lists the pending ADT events that need review by the hospitalist user associated with the client device, with associated metadata. In response to receiving the data object, the client device may generate and display a “to-do” list like that shown in FIG. 5A, including a list of patient names.

The method 1400 may further include, at 1420 by the at least one processor, outputting a first input object on the client device for user input indicating health conditions of the patient. For example, the client device may receive and display an admissions checklist as shown in FIG. 6 or FIG. 7.

The method 1400 may further include, at 1430 by the at least one processor, outputting a second input object on the client device for user input confirming at least one of an admission status of the patient or a discharge status of the patient. For example, the client device may receive and display and recommendation confirmation screen 800, as shown in FIG. 8, based on an association between the patient identified in the patient list with a hospitalist user of the client device.

The method 1400 may further include, at 1440 by the at least one processor, providing to the patient database an identifier of the selected patient, the user input indicating the health conditions, and the at least one of an admission status or the discharge status of the patient. For example, the client device may transmit the patient identifier, user input, and statuses to a DMI server for use in administration and management.

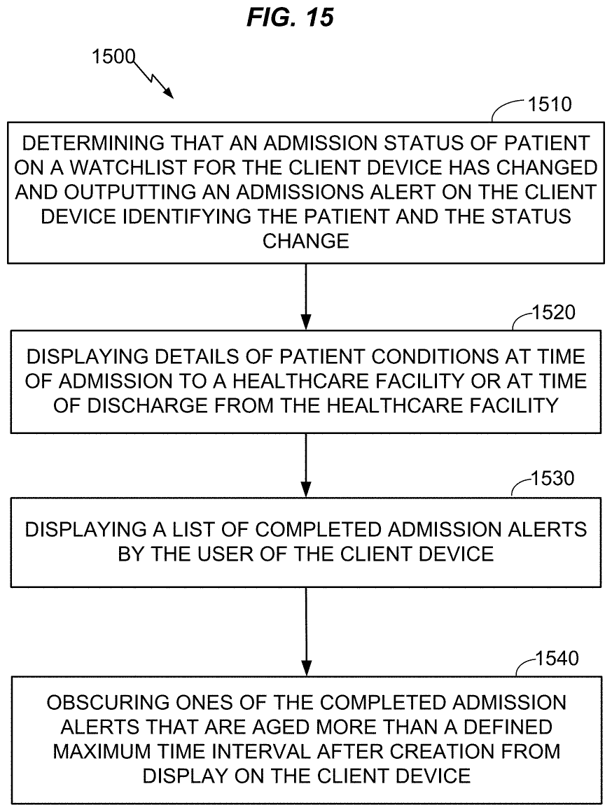

The method 1400 may include any one or more additional operations as described above and below herein. For example, FIGS. 15-17 illustrate optional additional operations that may be performed by one or more elements of the system 100 as part of, or in combination with, the method 1400. Each of these additional operations is not necessarily performed in every embodiment of the method, and the presence of any one of the operations does not necessarily require that any other of these additional operations also be performed.

For example, referring to FIG. 15, the method 1400 may further include any one or more of the further operations 1500. The further operations 1500 may include, at 1510 by at least one processor, determining that an admission status of patient on a watchlist for the client device has changed and outputting an admissions alert on the client device identifying the patient and the status change. For example, the client device may receive and output an alert when any change of ADT status for a patient in the hospital to which the hospitalist user is assigned occurs.

The further operations 1500 may include, at 1520 by the at least one processor, displaying details of patient conditions at time of admission to a healthcare facility or at time of discharge from the healthcare facility. For example, the client device may retrieve patient data and display it in a “to-do” list as shown in FIG. 5A, in a discharge screen 100 as shown in FIG. 10, or in a patient information screen as shown in FIG. 13A. The further operations 1500 may include, at 1530 by the at least one processor, displaying a list of completed admission alerts by the user of the client device. The further operations 1500 may include, at 1540 by the at least one processor, obscuring ones of the completed admission alerts that are aged more than a defined maximum time interval after creation from display on the client device.

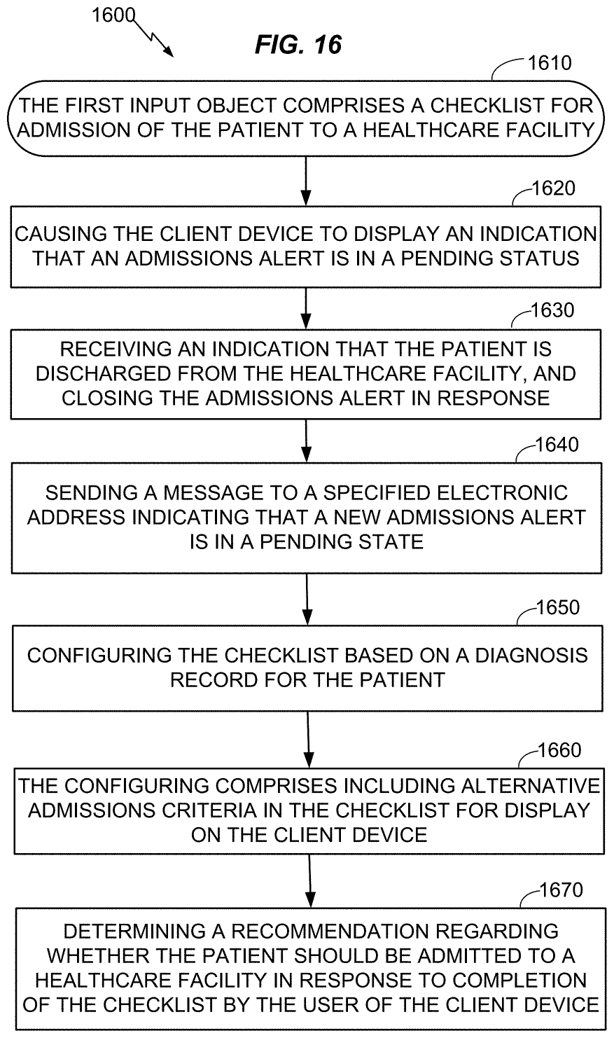

For example, referring to FIG. 16, the method 1400 may further include any one or more of the further operations 1600. Common to all the further operations 1600 is the feature 1610, wherein the first input object is, or includes, a checklist for admission of the patient to a healthcare facility.

The further operations 1600 may include, at 1620, by the at least one processor, causing the client device to display an indication that an admissions alert is in a pending status. Conversely, the further operations 1600 may include, at 1630, by the at least one processor receiving an indication that the patient is discharged from the healthcare facility and closing the admissions alert in response.

The further operations 1600 may include, at 1640, by the at least one processor, sending a message to a specified electronic address indicating that a new admissions alert is in a pending state.

The further operations 1600 may include, at 1650 by the at least one processor, configuring the checklist based on a diagnosis record for the patient. In a further aspect 1660, the configuring may include providing alternative admissions criteria in the checklist for display on the client device.

The further operations 1600 may include, at 1670 by the at least one processor, determining a recommendation regarding whether the patient should be admitted to a healthcare facility in response to completion of the checklist by the user of the client device.

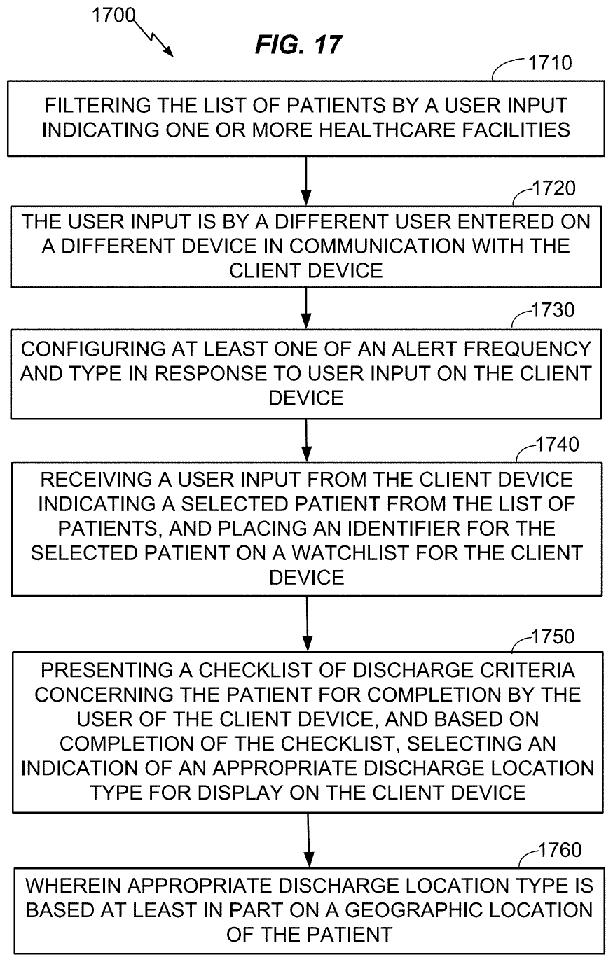

For example, referring to FIG. 17, the method 1400 may further include any one or more of the further operations 1700. The further operations 1700 may include, at 1710 by the at least one processor, filtering the list of patients by a user input indicating one or more healthcare facilities. In a further aspect of the filtering 1720, the user input is by a different user entered on a different device in communication with the client device.

The further operations 1700 may include, at 1730 by the at least one processor, configuring at least one of an alert frequency and type in response to user input on the client device. An example of a user input screen for a user-determined preference may be seen in screen 1350, FIG. 13B.

The further operations 1700 may include, at 1740 by the at least one processor, receiving a user input from the client device indicating a selected patient from the list of patients, and placing an identifier for the selected patient on a watchlist for the client device.

The further operations 1700 may include, at 1750, by the at least one processor presenting a checklist of discharge criteria concerning the patient for completion by the user of the client device, and based on completion of the checklist, selecting an indication of an appropriate discharge location type for display on the client device. In a further aspect 1760, the at least one processor bases the appropriate discharge location type at least in part on a geographic location of the patient.

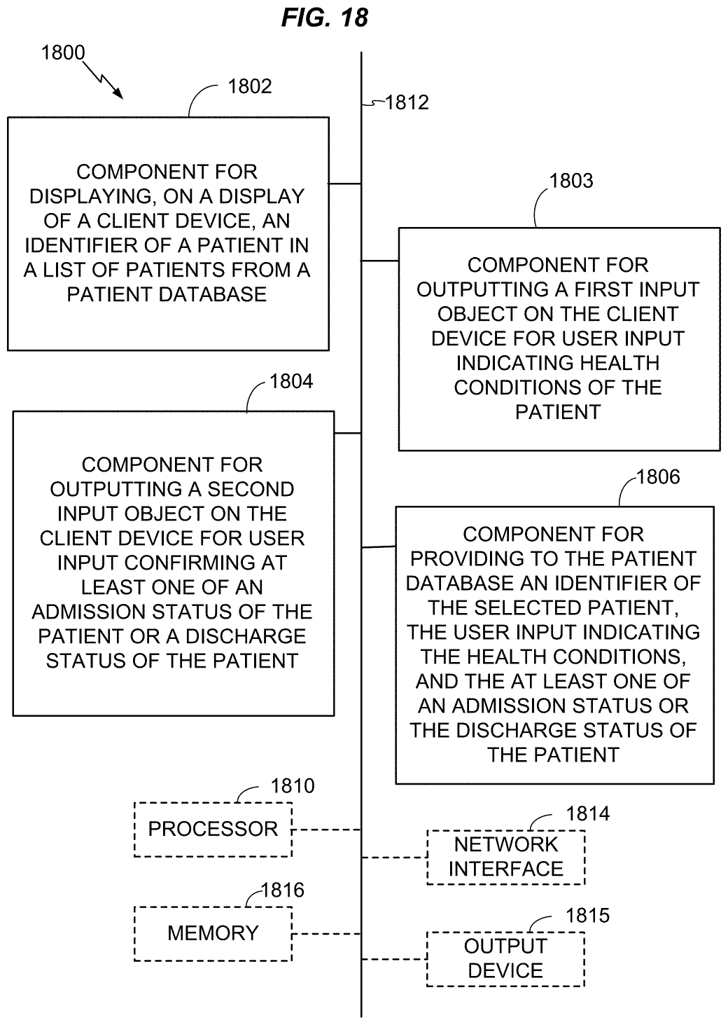

FIG. 18 is a conceptual block diagram illustrating components of an apparatus or system 1800 for configuring a client device to assist a user with managing patient conditions, treatments, admissions, and discharges as described herein, according to one embodiment. As depicted, the apparatus or system 1800 may include functional blocks that can represent functions implemented by one or more processors, software, or a combination thereof (e.g., firmware).

As illustrated in FIG. 18, the apparatus or system 1800 may comprise an electrical component 1802 for displaying on a display of a client device, an identifier of a patient in a list of patients from a patient database. The component 1802 may be, or may include, a means for said displaying. Said means may include the processor 1810 coupled to the memory 1816, to the output device 1815, and to the network interface 1814, the processor executing an algorithm based on program instructions stored in the memory. Such algorithm may include a sequence of more detailed operations, for example, receiving a message from the network interface and distributing the message to the application layer, testing the message for compliance with a protocol, verifying authenticity of the message, and sending the message to a module that interprets information in the message as including an identifier for presenting in a field of a graphical user interface screen for the output device, indicating an identifier of a patient, for example as shown in FIG. 1.

As illustrated in FIG. 18, the apparatus or system 1800 may comprise an electrical component 1803 for outputting a first input object on the client device for user input indicating health conditions of the patient. The component 1803 may be, or may include, a means for said outputting. Said means may include the processor 1810 coupled to the memory 1816, to the output device 1815, and to the network interface 1814, the processor executing an algorithm based on program instructions stored in the memory. Such algorithm may include a sequence of more detailed operations, for example, receiving an input checklist identifier, retrieving the input checklist data object from a memory based on the identifier, and sending the data object to a module that generates a checklist input screen, for example as shown in FIG. 6.

As illustrated in FIG. 18, the apparatus or system 1800 may comprise an electrical component 1804 for outputting a second input object on the client device for user input confirming at least one of an admission status of the patient or a discharge status of the patient. The component 1804 may be, or may include, a means for said outputting. Said means may include the processor 1810 coupled to the memory 1816, to the output device 1815, and to the network interface 1814, the processor executing an algorithm based on program instructions stored in the memory. Such algorithm may include a sequence of more detailed operations, for example receiving a data object containing an input object for user input indicating at least one of a recommendation for admission status of the patient or a discharge status of the patient, retrieving the recommendation data object from a memory based on the identifier, and sending the recommendation data object to a module that generates a recommendation input screen, for example as shown in FIG. 9.

As illustrated in FIG. 18, the apparatus or system 1800 may comprise an electrical component 1806 for providing to the patient database an identifier of the selected patient, the user input indicating the health conditions, and the at least one of an admission status or the discharge status of the patient. The component 1804 may be, or may include, a means for said providing. Said means may include the processor 1810 coupled to the memory 1816, to the output device 1815, and to the network interface 1814, the processor executing an algorithm based on program instructions stored in the memory. Such algorithm may include a sequence of more detailed operations, for example, saving user input data from the first input object, the second input object, and the recommendation data object in a computer memory, and providing the saved input data to a transport layer of the network interface for delivery to a specified address.

The apparatus 1800 may optionally include a processor module 1810 having at least one processor, in the case of the apparatus 1800 configured as a data processor. The processor 1810, in such case, may be in operative communication with the modules 1802-1806 via a bus 1812 or other communication coupling, for example, a network. The processor 1810 may initiate and execute the processes or functions performed by electrical components 1802-1806.

In related aspects, the apparatus 1800 may include a network interface module 1814 operable for communicating with a storage device over a computer network, and an output device 1815, for example, an LED or similar display and graphics controller.

In further related aspects, the apparatus 1800 may optionally include a module for storing information, such as, for example, a memory device/module 1816. The computer readable medium or the memory module 1816 may be operatively coupled to the other components of the apparatus 1800 via the bus 1812 or the like. The memory module 1816 may be adapted to store computer readable instructions and data for effecting the processes and behavior of the modules 1802-1806, and subcomponents thereof, or the processor 1810, or the method 1400 and one or more of the additional operations 1500, 1600 or 1700 described in connection with the method 1400. The memory module 1816 may retain instructions for executing functions associated with the modules 1802-1806. While shown as being external to the memory 1816, it is to be understood that the modules 1802-1806 can exist within the memory 1816.

The various illustrative logical blocks, modules, circuits, and algorithm steps described in connection with the aspects disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the application and design constraints imposed on the overall system. Skilled artisans may implement the described functionality in varying ways for each application, but such implementation decisions should not be interpreted as causing a departure from the scope of the present disclosure.

As used in this application, the terms “component”, “module”, “system”, and the like are intended to refer to a computer-related entity, either hardware, a combination of hardware and software, software, or software in execution. For example, a component may be, but is not limited to being, a process running on a processor, a processor, an object, an executable, a thread of execution, a program, and/or a computer or system of cooperating computers. By way of illustration, both an application running on a server and the server can be a component. One or more components may reside within a process and/or thread of execution and a component may be localized on one computer and/or distributed between two or more computers.

Program instructions may be written in any suitable high-level language, for example, C, C++, C#, JavaScript, or Java™, and compiled to produce machine-language code for execution by the processor. Program instructions may be grouped into functional modules, to facilitate coding efficiency and comprehensibility. It should be appreciated that such modules, even if discernable as divisions or grouping in source code, are not necessarily distinguishable as separate code blocks in machine-level coding. Code bundles directed toward a specific function may be considered to comprise a module, regardless of whether machine code on the bundle can be executed independently of other machine code. In other words, the modules may be high-level modules only.

Various aspects will be presented in terms of systems that may include several components, modules, and the like. It is to be understood and appreciated that the various systems may include additional components, modules, etc. and/or may not include all the components, modules, etc. discussed in connection with the figures. A combination of these approaches may also be used. The various aspects disclosed herein can be performed on electrical devices including devices that utilize touch screen display technologies and/or mouse-and-keyboard type interfaces. Examples of such devices include computers (desktop and mobile), smart phones, personal digital assistants (PDAs), and other electronic devices both wired and wireless.

In addition, the various illustrative logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. As used herein, a “processor” encompasses any one or functional combination of the foregoing examples.

Operational aspects disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

Furthermore, the one or more versions may be implemented as a method, apparatus, or article of manufacture using standard programming and/or engineering techniques to produce software, firmware, hardware, or any combination thereof to control a computer to implement the disclosed aspects. Non-transitory computer readable media can include but are not limited to magnetic storage devices (e.g., hard disk, floppy disk, magnetic strips . . . ), optical disks (e.g., compact disk (CD), digital versatile disk (DVD), BluRay™ . . . ), smart cards, solid-state devices (SSDs), and flash memory devices (e.g., card, stick). Of course, those skilled in the art will recognize many modifications may be made to this configuration without departing from the scope of the disclosed aspects.

In view of the exemplary systems described supra, methodologies that may be implemented in accordance with the disclosed subject matter have been described with reference to several flow diagrams. While for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methodologies described herein. Additionally, it should be further appreciated that the methodologies disclosed herein are capable of being stored on an article of manufacture to facilitate transporting and transferring such methodologies to computers.

The previous description of the disclosed aspects is provided to enable any person skilled in the art to make or use the present disclosure. Various modifications to these aspects will be clear to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

1. A system for verifying patient conditions, treatments, and admissions, the system comprising:
a patient database; and
a client application capable of communicating with the patient database, the application configured to:
display on a display of a client device a list of patients awaiting review, wherein the list of patients is received from the patient database;
receive, from a user, a selection of a patient from the list of patients;
request the user to input one or more conditions of the patient;
request the user to confirm an admission status as observation or inpatient; and
update the patient database with the inputted one or more conditions and admission status.

2. A method for configuring a client device to assist a user with managing patient conditions, treatments, admissions, and discharges, the method comprising:
displaying, by at least one processor on a display of a client device, an identifier of a patient in a list of patients from a patient database;
outputting, by the at least one processor, a first input object on the client device for user input indicating health conditions of the patient;
outputting, by the at least one processor, a second input object on the client device for user input confirming at least one of an admission status of the patient or a discharge status of the patient; and
providing, by the at least one processor to the patient database, an identifier of the selected patient, the user input indicating the health conditions, and the at least one of an admission status or the discharge status of the patient.

3. The method of claim 2, further comprising by the at least one processor determining that an admission status of patient on a watchlist for the client device has changed and outputting an admissions alert on the client device identifying the patient and the status change.

4. The method of claim 3, wherein the determining and the outputting the admissions alert are preconditions to performing all steps of claim 2.

5. The method of claim 2, further comprising by the at least one processor displaying details of patient conditions at time of admission to a healthcare facility or at time of discharge from the healthcare facility.

6. The method of claim 2, further comprising by the at least one processor displaying a list of completed admission alerts by the user of the client device.

7. The method of claim 6, further comprising by the at least one processor obscuring ones of the completed admission alerts that are aged more than a defined maximum time interval after creation from display on the client device.

8. The method of claim 2, wherein the first input object comprises a checklist for admission of the patient to a healthcare facility, and further comprising, by the at least one processor, causing the client device to display an indication that an admissions alert is in a pending status.

9. The method of claim 8, further comprising by the at least one processor receiving an indication that the patient is discharged from the healthcare facility, and closing the admissions alert in response.

10. The method of claim 8, further comprising by the at least one processor, sending a message to a specified electronic address indicating that a new admissions alert is in a pending state.

11. The method of claim 8, further comprising by the at least one processor configuring the checklist based on a diagnosis record for the patient.

12. The method of claim 11, wherein the configuring comprises providing alternative admissions criteria in the checklist for display on the client device.

13. The method of claim 8, further comprising by the at least one processor determining a recommendation regarding whether the patient should be admitted to a healthcare facility in response to completion of the checklist by the user of the client device.

14. The method of claim 2, further comprising by the at least one processor filtering the list of patients by a user input indicating one or more healthcare facilities.

15. The method of claim 14, wherein the user input is by a different user entered on a different device in communication with the client device.

16. The method of claim 2, further comprising configuring at least one of an alert frequency and type in response to user input on the client device.

17. The method of claim 2, further comprising by the at least one processor receiving a user input from the client device indicating a selected patient from the list of patients, and placing an identifier for the selected patient on a watchlist for the client device.

18. The method of claim 2, further comprising by the at least one processor presenting a checklist of discharge criteria concerning the patient for completion by the user of the client device, and based on completion of the checklist, selecting an indication of an appropriate discharge location type for display on the client device.

19. The method of claim 18, wherein appropriate discharge location type is based at least in part on a geographic location of the patient.

20. An apparatus for assisting a user with managing patient conditions, treatments, admissions, and discharges, the apparatus comprising a processor coupled to an electronic memory, the memory holding instructions that when executed by the processor cause the apparatus to perform:
displaying on a display of a client device, an identifier of a patient in a list of patients from a patient database;
outputting a first input object on the client device for user input indicating health conditions of the patient;
outputting a second input object on the client device for user input confirming at least one of an admission status of the patient or a discharge status of the patient; and
providing to the patient database an identifier of the selected patient, the user input indicating the health conditions, and the at least one of an admission status or the discharge status of the patient.
